(19) United States
(12) Patent Application Publication
(10) Pub. No.: US 2023/0270707 A1
(43) Pub. Date: Aug. 31, 2023

(54) ORAL LIQUID PHARMACEUTICAL COMPOSITIONS OF ISOTRETINOIN

(71) Applicant: IDRS Labs Private Limited, Bangalore (IN)

(72) Inventors: Mahendra R. Joshi, Bangalore (IN); Archana Khosa Kakkar, Chandigarh (IN); Vikram Prakash Gota, Navi Mumbai (IN); Shripad D. Banavali, Mumbai (IN); Girish Chinnaswamy, Mumbai (IN); Ravindhar Reddy Mandapati, Bangalore (IN); Mahaboob Basha Shaik, Prakasam (IN)

(21) Appl. No.: 18/310,501

(22) Filed: May 1, 2023

Related U.S. Application Data
(63) Continuation-in-part of application No. PCT/IN2021/051040, filed on Oct. 29, 2021.

(30) Foreign Application Priority Data
Nov. 1, 2020 (IN) 202041047657

(51) Int. Cl. A61K 31/203; A61K 47/24; A61K 47/14; A61K 47/10; A61K 47/26; A61K 47/44

(52) U.S. Cl. CPC A61K 31/203; A61K 47/24; A61K 47/14; A61K 47/10; A61K 47/26; A61K 47/44

(57) ABSTRACT

The present invention relates to new stable oral liquid pharmaceutical compositions comprising isotretinoin (13-cis retinoic acid), pharmaceutically accepted salts thereof, or other suitable isotretinoin compounds. The compositions are formulated in a lipid carrier, typically comprising phospholipids, and in embodiments comprising a substantial amount of one or more phosphatidylcholines. Such compositions can further comprise a surfactant component, a medium chain triglyceride component, an antioxidant/preservative component, or combination thereof. The present invention also provides the process of preparation of such compositions and methods of using such compositions in the treatment of various diseases/conditions.

### RELATED APPLICATIONS/PRIORITY

This patent application is a continuation-in-part of International Patent Application No. PCT/IN2021/051040 (published as WO 2022/091140), filed Oct. 29, 2021, which claims priority to Indian Patent Application IN202041047657, filed Nov. 1, 2020. This application claims the benefit of priority to, and incorporates by reference the entirety of, these referenced patent applications.

### FIELD OF THE INVENTION

This invention relates to new pharmaceutical compositions comprising an effective amount of one or more isotretinoin compounds, such as isotretinoin or one or more pharmaceutically acceptable salts of isotretinoin, typically formulated in, among other elements, an effective amount of a suitable and effective lipid carrier, which, in aspects, comprises phospholipids, such as, e.g., phosphatidylcholine. The present invention further relates to the processes for preparation of such compositions and for methods of using such compositions in the treatment of diseases, such as cancer, dermatologic diseases (e.g., acne), or for any other condition that is known to be subject to treatment with isotretinoin.

### BACKGROUND OF THE INVENTION

Isotretinoin (ISA), also known as 13-cis retinoic acid (13-CRA). is a Vitamin A derivative. Isotretinoin, its isomers, and analogs/derivatives thereof, can exhibit therapeutic efficacy in severe dermatological disorders such as cystic acne, cutaneous lupus erythematosus, psoriasis, and keratinization disorders, among other diseases/conditions.

Isotretinoin received FDA approval in 1982 for severe recalcitrant acne under the brand name ACCUTANE®, marketed by Hoffman La Roche as a suspension of isotretinoin encapsulated in soft gelatin capsule dosage form. Later, Sun Pharmaceuticals received approval for a hard gelatin capsule, using patented Lidose® technology with enhanced bioavailability, under the brand name ABSORICA®. The current recommended dose of isotretinoin is 0.5 to 1 mg/kg/day given in two divided doses with or without meals for 15 to 20 weeks. ABSORICA LD®, which received approval in 2019 is a low dose formulation with recommended dose of 0.4 to 0.8 mg/kg/day given in two divided doses with or without meals for 15 to 20 weeks.

Isotretinoin is known to modulate cell growth, induce cell differentiation as well as apoptosis and hence has proven efficacious against various cancers such as skin cancers, cutaneous T-cell lymphoma, acute promyelocytic leukemia, lung cancer, breast cancer, ovarian cancer, bladder cancer, kidney cancer, head and neck cancers when used alone or in combination with other therapeutic agents/methods.

Isotretinoin is used to treat neuroblastoma, the most common type of extracranial solid tumor in children. Neuroblastomas comprise 97% of the total sympathetic nervous system cancers and represents 7.8% of cancer in children younger than 15 years of age. Because of the aggressive nature and high likelihood of metastatic disease at diagnosis, neuroblastoma accounts for nearly 15% of all pediatric cancer fatalities. Treatment with isotretinoin in high-risk neuroblastoma reduces recurrence after high dose chemotherapy and stem cell transplant. Isotretinoin is given along with immunotherapy during the maintenance phase of the therapy. Isotretinoin therapy is recommended for around 6 months for the condition where it is taken twice a day for two weeks followed by two weeks off the medication.

As is evident from various studies, isotretinoin therapy is prescribed for pediatric as well as adults including geriatric patients. Oral route is the preferred mode of administration of therapeutics. However, pediatric patients, especially patients 10 years or younger, often find it difficult to swallow solid dosage forms such as tablets and capsules. Several barriers exist for children in swallowing capsules including anxiety, strong gag reflex, texture, size, and shape of capsule (Katherine K. Matthay, MD., Targeted Isotretinoin in Neuroblastoma: Kinetics, Genetics or Absorption, Clin Cancer Res. 2013 Jan. 15; 19(2): 311-313). Children who fear swallowing capsules are likely to be tense when attempting to do so, therefore making the process more difficult. This tension, particularly in the throat, neck and chest can make the child feel like they are having trouble breathing which may cause further anxiety. Children who are fussy eaters or who gag frequently on food and drink can often struggle with swallowing medicines (strong gag reflex). Further, the texture, size, shape of the capsule, and the nature of the coating can affect the ease of swallowing for children.

Further, pediatric patients often need lower doses to be administered based on the body weight and body surface area which is difficult to achieve with tablets or capsules. Cutting, crushing, or manipulating tablets or capsules to achieve pediatric doses may result in inaccurate dosing, potentially leading to increased adverse effects or decreased effectiveness. The same problems can result from rounding each dose to the nearest tablet or capsule.

Preclinical and clinical studies have suggested that maintenance of adequate 13-CRA plasma levels is crucial to drug activity (Reynolds et al. Response of neuroblastoma to retinoic acid in vitro and in vivo. Prog Clin Biol Res. 1991; 366:203-11). Vel's et al attempted to examine potential factors that might affect achieving the target plasma level of isotretinoin ≥2 μM including clinical features, dose regimen, metabolism, pharmacogenetics, and mode of administration. Young children, who were unable to swallow capsules, were administered isotretinoin after cutting the capsules, extracting out the contents and mixing the drug either with soft foods before oral administration, or directly administering via nasogastric tube. In such studies, 92.5% of patients who were able to swallow the capsules achieved the target Cmax as compared to only 55% of those who extracted the drug and mixed it with food or gave it via nasogastric tube. Thus, the lack of an age-appropriate formulation clearly has a detrimental effect on the attainable plasma levels of isotretinoin (Katherine K. Matthay, MD., Targeted isotretinoin in neuroblastoma: Kinetics, Genetics or Absorption, Clin Cancer Res. 2013 Jan. 15; 19(2): 311-313). Therefore, to achieve accurate and flexible dosing, improved therapeutic outcome, enhanced patient compliance especially for patients suffering from dysphagia (i.e., difficulty in swallowing) and for administration via the nasogastric tube, effective oral dosage forms of isotretinoin will be required.

However, there are numerous challenges to developing such a formulation and, to date, no product containing such a formulation is available in most major markets. Isotretinoin is a highly lipophilic molecule which is practically insoluble in water and is highly sensitive to light and oxygen. Further, isotretinoin undergoes reversible isomeric conversion which can have an impact on the assay of isotretinoin in the formulation. Owing to these issues it is very difficult to formulate a solution form of the molecule. Again, despite the attractiveness of oral formulations, there is no oral solution dosage form of isotretinoin available in the market currently. This does not mean to imply that attempts to develop such formulations and related technologies have not been previously made. In fact, numerous patent disclosures suggest that there have been several attempts at developing new isotretinoin formulations that can address the known shortcomings of isotretinoin drugs, even by very successful pharmaceutical companies.

For example, U.S. Pat. No. 9,078,925 discloses an oral pharmaceutical composition comprising isotretinoin, a lipophilic carrier that comprises fatty acid esters (polyol ester of medium chain fatty acid selected from esters and mixed esters of glycerol, propylene glycol, polyglycerol, and polyethylene glycol, along with medium chain fatty acids), fatty acids (C6-C20 saturated, mono- and di-unsaturated fatty acids), fatty alcohols (C6-C20 saturated, mono, di-unsaturated alcohols), vegetable oil (kernel oil, almond oil, groundnut oil, olive oil, soybean oil, safflower oil, sunflower oil, palm oil, sesame oil, canola oil, or corn oil) and a hydrophilic carrier (monohydric alcohols, glycols, polyols, and glycerols).

U.S. Pat. No. 9,999,606 demonstrates the higher bioavailability of an oral pharmaceutical composition of isotretinoin over a capsule comprising a semi-solid suspension of isotretinoin. In the formulations described in this disclosure, isotretinoin is in the form of gel, dispersion, suspension, or emulsion is adsorbed onto a carrier substrate selected from the group consisting of lactose, microcrystalline cellulose, calcium phosphate, dextrin, dextrose, sucrose, mannitol, maltodextrin, and sodium aluminosilicate to form solid particles, powder, or granules.

U.S. Pat. No. 8,367,102 and related WO2002024172 (Galephar) discloses semi-solid oral pharmaceutical compositions of isotretinoin containing at least two excipients, one of them being hydrophilic (i.e., having an HLB value superior to or equal to 10), the other being an oily vehicle. The '172 PCT application describes that the high HLB hydrophilic component of the formulation is preferably composed of glycerol macrogolglycerides. Actual formulations exemplified in the '172 PCT application include lauroyl macrogol-32 glycerides (Gelucire®44/14, Gattefosse) and stearoyl macrogol-32 glycerides (Gelucire®50/13, Gattefosse) in combination with soyabean oil and possibly other agents (e.g., Labrafil®M1944 CS 132 and Mygliol®320). Such compositions are in a semi-solid form at room temperature and can be used to, e.g., fill capsule dosage forms. Such formulations are described as being provided for the treatment of acne, hypertrophic lupus erythematosus, basal cell carcinoma, and squamous cell carcinoma, and possibly other purposes.

U.S. Pat. No. 7,781,489 discloses a water-in-oil microemulsion comprising isotretinoin as an active ingredient, a phospholipid emulsifier, and sodium hyaluronate, wherein the phospholipid emulsifier is phosphatidylcholine or soya lecithin.

US 2018318245 discloses a pharmaceutical composition comprising isotretinoin, PEG 400, methyl paraben, propyl paraben, PEG 4000, and BHT wherein the pharmaceutical composition is formulated as a gel, ointment, lotion, emulsion, or cream.

U.S. Pat. No. 6,740,337 discloses a bioavailable capsule composition of isotretinoin.

WO 0000179 discloses a solid dispersed preparation for poorly water-soluble drugs prepared by dissolving or dispersing the drugs in an oil, a fatty acid, or a mixture thereof, mixing the solution or dispersion in a water-soluble polyol matrix and drying the mixture.

U.S. Pat. No. 7,435,427 discloses capsules comprising a semi-solid suspension of isotretinoin containing at least two lipidic excipients, one having an HLB value equal to or greater than 10 and the other being an oily vehicle.

IN813CHE2011A (issued as IN333223B) discloses self-nanoemulsifying drug delivery system (SNEDDS) for isotretinoin intended to achieve a rapid drug release and enhance dissolution. The formulations of the '011 IN application include a fatty material, which may be selected from the group consisting of lecithin, triglycerides, wax, phospholipids, vegetable oils, essential oil, animal oil, mineral oil, plant oil, medium chain triglycerides, fatty acid esters, glycerol oleate, glycerol triacetate and mixtures thereof (including peppermint oil, sesame oil, refined soya oil, CRODAMOL GTCC, triacetin, LABRAFIL M, LABRAFIL CS and mixtures thereof, with essential oil being preferred). The fatty material is indicated as being present in an amount of 15-40%, typically 25-35%. The composition further comprises a surfactant/co-surfactant surfactant system (which may include sorbitan fatty acid esters, polysorbate derivatives, polyoxyethylene sorbitan fatty acid esters, derivatives of lecithin, propylene glycol esters, fatty acid esters of propylene glycol, and fatty acid esters of glycerol, polyethylene glycol, and mixtures thereof and exhibits an hydrophile lipophile balance (HLB) value in the range of 3 to 16, with polysorbate-80 being preferred and the surfactant system makes up 30-55% of the composition, preferably 40-55% of the composition, and notably a greater portion of the composition than the fatty material.

WO 2010134047 (Ranbaxy) discloses liquid pharmaceutical solutions that include isotretinoin or pharmaceutically acceptable salts thereof and a lipophilic carrier or a combination of lipophilic/hydrophilic carriers and describe the use thereof in capsules or other dosage forms. The lipophilic carrier may be fatty acid esters, fatty acids, fatty alcohols, vegetable oil (including, among several others, soybean oil), or a combination thereof. The hydrophilic carrier may be monohydric alcohols, glycols, polyols, glycerols, or combination thereof. A consistent element of the formulations described with any specificity and tested in the '047 PCT application include caprylic/capric triglyceride, dicaprylate/dicaprate, or propylene glycol monolaurate components (with caprylic/capric triglyceride components being most used).

Despite these numerous disclosures and associated development programs/efforts, alternative oral formulations of isotretinoin remain lacking among approved drugs and, accordingly, the problems described herein have yet to be adequately addressed. Indeed, many of the above-described patent applications were abandoned/not maintained suggesting that the concepts described therein ultimately lacked sufficient merit to be developed as useful pharmaceutical products. The large number of attempts at arriving at such solutions with limited, little, or no success evidence that the development of effective isotretinoin formulations that can overcome such known problems requires truly inventive ingenuity.

### CONSTRUCTION, TERMS, AND ACRONYMS

This section offers guidelines for reading this disclosure. The intended audience for this disclosure (“readers”) are persons having ordinary skill in the practice of technologies discussed or used herein. Readers may also be called “skilled persons,” and such technologies called “the art.” Terms such as “understood,” “known,” and “ordinary meaning,” refer to the general knowledge of skilled persons.

The term “uncontradicted” means not contradicted by this disclosure, logic, or plausibility based on knowledge of skilled persons.

Disclosed here are several different but related exemplary aspects of the invention (referred also to as, e.g., “cases,” “facets,” or “embodiments”). The invention encompasses all aspects, as described individually and as can be arrived at by any combination of such individual aspects. The breadth and scope of the invention should not be limited by any exemplary embodiment(s). No language in this disclosure should be construed as indicating any element/step is essential to the practice of the invention unless such a requirement is explicitly stated. Uncontradicted, any aspect(s) can be combined with any other aspect(s).

Uncontradicted, all technical/scientific terms used here generally have the same meanings as commonly understood by skilled persons, regardless of any narrower examples or descriptions provided here (including any term introduced initially in quotations). However, aspects characterized by the inclusion of elements, steps, etc., associated with specific descriptions provided here are distinct embodiments of the invention. Uncontradicted, disclosure of any aspect using known terms, which terms are narrowed by example or otherwise in this disclosure, implicitly discloses related aspects in which such terms are alternatively interpreted using the broadest reasonable interpretation of skilled persons.

Uncontradicted, “or” means “and/or” here, regardless of any occasional inclusion of “and/or” (e.g., phrases such as “A, B, or C” and “A, B, and/or C” simultaneously disclose aspects including (1) all of A, B, and C; (2) A and C; (3) A and B; (4) B and C; (5) only A; (6) only B; and (7) only C (and also support sub-groupings, such as “A or B,” “A or C,” etc.)).

Uncontradicted, “also” means “also or alternatively.” Uncontradicted, “here” & “herein” mean “in this disclosure.” The term “i.a.” means “inter alia” or “among other things.” “Also known as” is abbreviated “aka” or “AKA.” “Elsewhere” means “elsewhere herein.”

For conciseness, symbols are used where appropriate. E.g., “&” is used for “and,” & “˜” for “about.” Symbols such as < and > are given their ordinary meaning (e.g., “≤” means “less than or equal to” & “≥” means “greater than or equal to”). A slash “/” can represent “or” (“A/B” means “A or B”) or identify synonyms of an element, as will be clear from context.

The inclusion of “(s)” after an element or a step indicates that ≥1 of such an element is present, step performed, and the like. E.g., “element(s)” means both 1 element or ≥2 elements, with the understanding that each thereof is an independent aspect of the invention. Uncontradicted, where an element is only provided in standard plural form (e.g., “compositions” as opposed to composition(s)), the reader should interpret such disclosure as encompassing a single composition as if presented as “composition(s).” Uncontradicted, any aspect disclosed herein in with an element or step expressed in the singular provides implicit support for a corresponding embodiment in which the element(s)/step(s) are present/performed in the plural (two or more), and vice versa.

Use of the abbreviation “etc.” (or “et cetera”) and similar terms (e.g., “and the like”) in association with a list of elements/steps means any or all suitable combinations of the recited elements/steps or any known equivalents of such recited elements/steps for achieving the function(s) of such elements/steps that are known in the art. Uncontradicted, terms such as “and combinations,” or “or combinations” (e.g., “and combinations thereof” or “or mixtures thereof”) regarding listed elements (e.g., components or ingredients/agents)/steps means any or all possible/suitable combinations of such elements/steps.

Aspects may be described as “suitable” for use(s) disclosed herein. Uncontradicted, terms such as “suitability” means acceptable or appropriate for performing a particular function/achieving particular state(s)/outcome(s), and typically means effective, practical, and non-deleterious/harmful in the context the term is used. E.g., uncontradicted, the term “suitable” means appropriate, acceptable, or in contexts sufficient, or providing at least generally or substantially all of an intended function, without causing or imparting significant negative/detrimental impact.

Uncontradicted, heading(s) (e.g., “Construction, Terms . . . ”) and subheadings are included for convenience and do not limit the scope of any aspect(s). Uncontradicted, aspect(s), step(s), or element(s) described under one heading or in any one section can apply to other aspect(s) or step(s)/element(s) here.

Ranges of values are used to represent each value falling within such range that are within an order of magnitude of the smallest endpoint of the range without having to explicitly write each value of the range. E.g., a recited range of 1-2 implicitly discloses each of 1.0, 1.1, 1.2, . . . 1.9, and 2.0 and 10-100 implicitly discloses each of 10, 11, 12, . . . 98, 99, and 100). Uncontradicted, all ranges include the range's endpoints, regardless of how a range is described. E.g., “between 1-5” includes 1 and 5 in addition to 2, 3, and 4 (and all numbers between such numbers within an order of magnitude of such endpoints, e.g., 1.0, 1.1, . . . 4.9, and 5.0). For the avoidance of doubt, any number within a range, regardless of the order of magnitude of the number, is covered by the range (e.g., a range of 2-20 covers 18.593).

Terms of approximation (e.g., “about,” “˜,” or “approximately”) are used (1) to refer to a set of related values or (2) where a precise value is difficult to define (e.g., due to limits of measurement). Uncontradicted, all exact values provided here simultaneously/implicitly disclose corresponding approximate values and vice versa (e.g., disclosure of “about 10” provides explicit support for the use of 10 exactly in such aspect/description). Ranges described with approximate value(s) include all values encompassed by each approximate endpoint, regardless of presentation (e.g., “about 10-20” has the same meaning as “about 10-about 20”). The scope of value(s) encompassed by an approximate term typically depends on the context of the disclosure, criticality or operability, statistical significance, understanding in the art, etc. In the absence of guidance here or in the art for an element, terms such as “about” when used in connection with an element should be interpreted as ±10% of the indicated value(s) and implicitly concurrently disclosing ±5%, ±2%, ±1%, and ±0.5% of such value(s).

This disclosure includes aspects associated with particular characteristics, such as amounts of components (or ranges thereof), In cases, several such characteristics of varying scope may be provided. Readers will understand that each such characteristic can be associated with particular properties that distinguish such aspects from other aspects, and, accordingly, each such range can be viewed as critical to a particular aspect of the invention, even if the associated results, properties, functions, etc., associated with such aspects are not directly communicated in association with such characteristics.

Lists of aspects, elements, steps, and features are sometimes employed for conciseness. Unless indicated, each member of each list should be viewed as an independent aspect. Each aspect defined by any individual member of a list can have, and often will have, nonobvious properties vis-a-vis aspects characterized by other members of the list.

Uncontradicted, the terms “a” and “an” and “the” and similar referents encompass both the singular and the plural form of the referenced element, step, or aspect. Uncontradicted, terms in the singular implicitly convey the plural and vice versa herein (in other words, disclosure of an element/step implicitly discloses corresponding use of such/similar elements/steps and vice versa). Hence, e.g., uncontradicted, a passage regarding an aspect including X step supports a corresponding aspect including several X steps. Uncontradicted, any mixed use of a referent such as “a” in respect of one element/step or characteristic and a phrase like “one or more of” with respect to another element/step or characteristic in a paragraph, sentence, aspect, or claim, does not change the meaning of such referents. Thus, for example, if a paragraph describes a composition comprising “an X” and “one or more Ys,” the paragraph should be understood as providing disclosure of “one or more Xs” and “one or more Ys.”

“Significant” and “significantly” mean results/characteristics that are statistically significant using ≥1 appropriate test(s)/trial(s) in the given context (e.g., p≤0.05/0.01). “Detectable” means measurably present/different using known detection tools/techniques. The acronym “DOS” (or “DoS”) means “detectable(ly) or significant(ly).”

Uncontradicted, any value here that is not accompanied by a unit of measurement (e.g., a weight of 50 or a length of 20), any previously provided unit for the same element/step or the same type of element/step will apply, or, in cases where no such disclosure exists, the unit most commonly used in association with such an element/step in the art will apply.

Uncontradicted, the terms “including,” “containing,” “comprising,” and “having” mean “including, but not limited to” or “including, without limitation.” Uncontradicted, use of terms such as comprising and including regarding elements/steps means including any detectable number or amount of an element or including any detectable performance of a step/number of steps (with or without other elements/steps).

For conciseness, description of an aspect “comprising” or “including” an element, with respect to a collection/whole (e.g., a system, device, or composition), uncontradicted, implicitly provides support for any detectable amount/number or ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the whole/collection being made up of the element, or essentially all of the whole/collection being made up of the element (i.e., that the collection consists essentially of the referenced element). Similarly, uncontradicted, a method described as including a step with respect to an effect/outcome implicitly provides support for the referenced step providing ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the effect/outcome, representing ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the steps/effort performed, or both. Explicit listing of percentages of elements/steps in connection with aspects does not limit or contradict such implicit disclosure described in this paragraph.

Uncontradicted, terms such as “comprising” when used in connection with a step of a method provide implicit support for performing the step once, ≥2 times, or until an associated function/effect is achieved.

Uncontradicted, the term “one” means a single type, single iteration/copy/thing, of a recited element or step, or both, which will be clear from context. For example, the referent “one” used with a component of a composition can refer to one type of element (which may be present in numerous copies, as in the case of an ingredient in a composition), one unit of the element, or both. Similarly, “one” component, a “single” component, or the “only component” of a system typically means 1 type of element (which may be present in numerous copies), 1 instance/unit of the element, or both. Further, “one” step of a method typically means performing one type of action (step), one iteration of a step, or both. Uncontradicted, a disclosure of “one” element provides support for both, but uncontradicted, any claim to any “one” element means one type of such an element (e.g., a component of a composition/system).

Uncontradicted, the term “some” means ≥2 copies/instances or ≥5% of a listed collection/whole is, or is made up of, an element. Regarding methods, uncontradicted “some” means ≥5% of an effect, effort, or both, is made up of or is attributable to a step (e.g., as in “some of the method is performed by step Y”) or indicates a step is performed ≥2 times (e.g., as in “step X is repeated some number of times”).

Uncontradicted, “predominately,” “most,” or “mostly,” means detectably >50% (e.g., mostly comprises, predominately includes, etc., mean >50%) (e.g., a system that mostly includes element X is composed of >50% of element X). The term “generally” means ≥75% (e.g., generally consists of, generally associated with, generally comprises, etc., means ≥75%) (e.g., a method that generally consists of step X means that 75% of the effort or effect of the method is attributable to step X). “Substantially” or “nearly” means ≥95% (e.g., nearly all, substantially consists of, etc., mean≥95%) (e.g., a collection that nearly entirely is made up of element X means that at least 95% of the elements in the collection are element X). Terms such as “generally free” of an element or “generally lacking” an element mean comprising ≤˜25% of an element and terms such as “substantially free” of an element mean comprising ≤˜5% of an element. Uncontradicted, terms such as “main,” “principal,” etc., when used to refer to an element or step (e.g., the “main ingredient”), mean in the greatest amount or of the greatest effect, as applicable, compared with other, usually similar, elements or steps.

In certain embodiments describing API(s), excipient(s), or both present in amounts of “at least” or “greater than” a given amount or, e.g., present in amounts of “no more than” or “no greater than” or “less than” a given amount, the reader should interpret such disclosure as disclosing, e.g., encompassing and explicitly including, such undefined low or high amount(s) ranging to the opposite amount (high or low) that is maximally/minimally therapeutically effective, typically suitable, or both. For example, use of the phrase “at least” (and similar descriptors) in connection with an amount of a component of a formulation or of an entire formulation/composition can be interpreted as at least the amount described but that is no more than a maximally suitable or therapeutically effective amount (in the individual or in a population, such as determined in a clinical study). Similarly, phrases such as “less than” (and similar descriptors) an indicated amount can be interpreted referring to an amount that is still suitable (including, where appropriate, no amount, e.g., 0 units of the indicated component) or therapeutically effective (e.g., an amount that results in a DOS result in a significant number of individuals in a well-controlled and adequate study), but is less than the indicated amount.

Constituents herein are typically present in “effective amounts,” and uncontradicted, any described class/type of, e.g., a composition or component/element thereof, such as an active ingredient or excipient (often referred to as a “component” herein—e.g., a “buffer component” may include one or more buffers) or specific excipient, or, e.g., in certain aspects active pharmaceutical ingredient(s) (API(s)) is understood to be present in the associated composition/formulation in an effective amount, which generally means, in this context, an amount that is effective for the described function(s) associated with the excipient/API or composition (it being understood that some excipient or API compound(s)/ingredient(s) exhibit more than one effect). E.g., a tonicity agent will be understood to be present in a composition/formulation in an amount that is effective to impart an indicated tonicity effect, a tonicity effect that is required for suitability of the composition, or an effect that imparts a detectable or significant tonicity effect on a composition (with respect to a comparator composition lacking the compound(s)/ingredient(s)). Uncontradicted, any step performed, function of a component, etc., is to be understood as being applied effectively, such as in an effective amount, which generally means, in this context, an amount that is effective for the described function(s) associated with the component, device, step, etc. Efficacy in this respect can mean, and, uncontradicted, should be interpreted to implicitly disclose efficacy in terms of (1) in a treated subject, (2) in a majority of subjects in a population, (3) in a statistically significant number of subjects in a population, (4) generally all subjects in a population, (5) substantially all subjects in a population, or (6) in a statistically significant number of or more of a typical or average subject of the class of subjects to be treated in the condition (treatment in this sense encompassing any administration, application, etc., whether for therapeutic reasons or prophylactic/maintenance reasons).

Uncontradicted, any aspect described with respect to an optionally present element(s)/step(s) also provides implicit support for corresponding aspect(s) in which one, some, most, generally all, nearly all, essentially all, or all of such element(s) are lacking/step(s) not performed, in respect of the relevant aspect. E.g., disclosure of a system comprising element X implicitly also supports a system lacking element X.

Uncontradicted, changes to tense or presentation of terms (e.g., using “comprises predominately” in place of “predominately comprises”) do not change the meaning of the corresponding term/phrase.

Uncontradicted, all methods provided here can be performed in any suitable order regardless of presentation (e.g., a method comprising steps A, B, and C, can be performed in the order C, B, and A; B and A and C simultaneously, etc.). Uncontradicted, elements of a composition can be assembled in any suitable manner by any suitable method. In general, any methods and materials similar or equivalent to those described here can be used in the practice of embodiments. Uncontradicted, the use of ordinal numbers such as “first,” “second,” “third,” etc. is to distinguish respective elements rather than to denote a particular order of those elements.

Uncontradicted, terms and phrases such as “suitable” and “suitable for” typically refer to, as applicable, methods or devices, which are generally safe and effective for application in the relevant context in which they are described, having sufficient strength/potency, quality, and safety for their intended application (e.g., where applicable for self-treatment of a condition), as may be judged by relevant experts, consumer approval/surveys, or regulatory authority review, or as established by, e.g., scientific measures, commercial success, expert opinions (e.g., medical opinions), one, two, or more well controlled and adequate clinical studies, adequately powered preclinical or consumer testing, and the like. Components, elements, and steps described as “suitable” should be interpreted in a similar manner.

“Pharmaceutical suitability”, “pharmaceutically suitable”, and the like, are phrases typically used to refer to compositions that are safe and effective for pharmaceutical administration and application, having sufficient potency, purity, strength, quality, and safety for pharmaceutical application, in cases specifically to the eye, as may be judged by regulatory authority review, and as established by, e.g., one or more well controlled and adequate clinical studies performed in compliance with generally prevailing regulatory authority standards. Uncontradicted, a description of “suitability” implicitly means that the referenced element, step, etc., is pharmaceutically suitable or otherwise medically suitable (e.g., safe and effective as determined by proper nonclinical/clinical testing).

Except where explicitly indicated or clearly indicated by context, “improved” herein means “increased.” In aspects, “improved” means “reduced,” such as with respect to the toxicity of a composition. Uncontradicted, terms such as “enhanced,” “improved,” and the like are used synonymously.

Uncontradicted, the word “exemplary” means “serving as an example, instance, or illustration.” The following detailed description is merely exemplary in nature and is not intended to limit application and uses. Any embodiment described herein as “exemplary” is not necessarily to be construed as preferred or advantageous over other embodiments.

Uncontradicted, any elements, steps, components, or features of aspects and all variations thereof, etc., are within the scope of the invention.

Elements associated with a function can be described as “means for” performing a function in a composition/device/system or a “step for” performing a part of a method, and parts of this disclosure refer to “equivalents,” which means equivalents known in the art for achieving a referenced function associated with disclosed mean(s)/step(s). However, no element of this disclosure or claim should be interpreted as limited to a “means-plus-function” construction unless such intent is clearly indicated by the use of the terms “means for” or “step for.” Terms such as “configured to” or “adapted to” do not indicate “means-plus-function” interpretation, but, rather, describe element(s)/step(s) configured to, designed to, selected to, or adapted to achieve a certain performance, characteristic, property, etc. using teachings provided here or in the art.

Uncontradicted, concentrations of ingredients/components of compositions/formulations herein are provided in weight percent concentration (wt. %). Thus, uncontradicted any reference to a percentage of an ingredient/component in a composition is made with reference to the weight percentage of that ingredient/component in the composition.

All references (e.g., publications, patent applications, and patents) cited herein are hereby incorporated by reference as if each reference were individually and specifically indicated to be incorporated by reference and set forth in its entirety herein. Uncontradicted, any suitable principles, methods, or elements of such references (collectively “teachings”) can be combined with or adapted to aspects. However, citation/incorporation of patent documents is limited to the technical disclosure thereof and does not reflect any view regarding the validity, patentability, etc., thereof. In the event of any conflict between this disclosure and the teachings of such documents, the content of this disclosure controls regarding aspects of the invention. Numerous references are cited here to concisely incorporate known information and aid skilled persons in putting aspects into practice. While efforts have been made to include the most relevant references for such purposes, readers will understand that not every aspect of every cited reference will apply to every aspect of the invention.

All original claims contained in this disclosure when filed are incorporated into this specification as if they were a part of the description.

### SUMMARY OF THE INVENTION

The embodiment(s) of the invention described and claimed herein have many attributes and aspects including, but not limited to, those set forth in, e.g., described or referenced in, this Summary. This Summary of the Invention (“Summary”) is not intended to be all-inclusive, and the scope of the invention is not limited to or by the aspects, features, elements, or embodiments provided in this Summary, which is included for illustrative purposes only and not restriction. Any of the aspects described under this section can be combined with any other aspect described in this section or with any other aspect of this disclosure.

The present invention discloses, i.a., stable oral liquid dosage forms of isotretinoin (13-CRA) solubilized in a lipid carrier, which can comprise, generally consist of, substantially consist of, or consist of/be a phospholipid carrier, leading to a solution form that can provide accurate and flexible dosing combined with desired therapeutic effects. In aspects, the compositions provided by this invention can be provided in concentrated form where the required dose can be delivered in small volumes which is desirable for patient compliance, particularly in special populations, such as pediatric patients. Further, in aspects, the compositions can be administered as such or mixed with milk, water and/or soft foods before administration.

The compositions of the invention can be present in any suitable form. In aspects, the form does not comprise a gel, a suspension, or an emulsion at room temperature. In one embodiment, the oral liquid pharmaceutical compositions of isotretinoin compound(s) of the invention are presented in solution form.

Oral liquid dosage forms are homogenous mixtures where the active ingredient(s) typically is/are either dissolved or suspended in a suitable vehicle or carrier. These dosage forms can offer advantages over solid oral dosage forms such as tablets and capsules in terms of providing flexible dosing based on body weight or body surface area, better patient compliance, suitability for pediatric and geriatric populations. Further, these dosage forms provide faster onset of action as well as more reproducible bioavailability.

Solutions are liquid dosage forms where the active ingredient is dissolved in a suitable vehicle resulting in a monophasic system. Solutions offer advantages over other liquid dosage forms as it results in accuracy of dosage as phase separation is not an issue. Further, the onset of action is faster, and it minimizes inter subject pharmacokinetic variability which is desired in highly variable molecules such as isotretinoin.

In a specific implementation, the stable oral liquid pharmaceutical composition includes a therapeutically effective amount of isotretinoin, a phospholipid carrier system/solubilizing component, and an effective amount of at least one pharmaceutically acceptable excipient (e.g., an antioxidant). In aspects, most, generally all, substantially all, essentially all, or all of the composition is composed of just these factors/elements. In other aspects, compositions comprise one or more additional elements, such as two, three, or more excipients, which can be, e.g., a surfactant/surfactant system, a preservative, etc.

In one embodiment of the invention, phospholipid(s) of a composition of the invention, are derived from soybean, eggs, or other known and suitable source of phospholipids, or are synthetic in nature, or the composition comprises a combination thereof. In aspects, the phospholipid compositions are either generally/entirely synthetic in origin or are from modified compositions of originally natural sources such as those mentioned herein (e.g., wherein the phospholipid(s) therein are concentrated, derivatized, modified, etc.).

In another embodiment of the invention the phospholipid comprises, primarily comprises, generally consists of, substantially consists of, or is, but not limited to, one or more phosphatidylcholine compound(s)/agent(s) (PC(s)). In embodiments a component of a formulation or an ingredient used in the manufacture of a formulation comprises an amount of at least one suitable phosphatidylcholine (“PC”), which along with the 13-CRA compound(s) of the formulation can be dissolved in a suitable amount of an effective and suitable solvent system which can be, e.g., a system that comprises or is composed of lipophilic carrier, hydrophilic carrier, emulsifying agents, or mixtures thereof.

In another implementation, the compositions may further include effect number(s) and amount(s) (number/amount) of one or more surfactants, antioxidants, chelating agents, sweeteners, flavors, preservatives, colors, or mixtures thereof.

In one implementation the pharmaceutical compositions of the present invention can be further filled in to capsules directly or after adsorbing it on a suitable solid carrier.

According to aspects, the invention provides stable, liquid pharmaceutical compositions comprising (1) a therapeutically effective amount of an isotretinoin compound (e.g., isotretinoin, a suitable/effective salt thereof, a suitable/effective analog/derivative thereof, or a salt thereof, or a suitable and effective mixture of any or all thereof); (2) a solubilizing component that is at least mostly composed of mixture of (a) phosphatidylcholine(s) and (b) a second solubilizing agent that is at least mostly composed of (I) a medium chain triglyceride (MCT) or mixture thereof, (II) a high oleic acid content oil comprising about 25-90% oleic acid, (III) a propylene glycol, or (IV) a combination thereof, wherein the amount of phosphatidylcholine in the solubilizing component is greater than the amount of the second component, the phosphatidylcholine makes up at least 25% of the total composition, or both conditions are met; and (3) a stabilizer component, comprising an effective amount of one or more stabilizing agents, wherein the one or more stabilizing agents comprise effective number(s)/amount(s) of one or more antioxidants, surfactants, medium chain triglycerides, or mixtures thereof, and, further, wherein the composition is a liquid at room temperature under ordinary conditions. Ordinary conditions mean conditions that would normally apply to a stored pharmaceutical product, such as temperature conditions (e.g., typically, generally only, substantially only, or only being maintained at about 2-55° C., such as 2.5-50° C., 2-50° C., 2.5-55° C., or ranges of temperature reflecting refrigerated conditions (e.g., +/−5%, 10%, 15%, or 20% of about 5° C.) or typical ambient temperature conditions (e.g., 5-60 or 10-50° C.) or room temperature conditions (e.g., +/−5%, 10%, or 15% of about 20 or about 25° C.) or conditions reflecting a combination thereof, and ordinary pressure conditions (e.g., +/−15%, 10%, 5%, or 2.5% of atmospheric pressure), and the like, which readers will be able to determine based on knowledge in the art as being suitable for the active pharmaceutical ingredient (here, isotretinoin compound(s)), the other elements of the formulation, and applicable condition(s) of intended use, storage, transport, etc. While conditions outside of such ordinary conditions may lead a formulation to no longer be a liquid, according to aspects, under such conditions a composition of the invention will mostly, generally, substantially, essentially, or entirely be in the form of a liquid (e.g., a liquid solution form).

Compositions can comprise any suitable isotretinoin compound(s). In aspects, the isotretinoin compounds comprise, mostly comprise, generally consist of, consist essentially of, substantially consist of, or consist of isotretinoin or one or more pharmaceutically acceptable salts thereof. In aspects, the isotretinoin compound comprises one or more suitable analogs or derivatives of isotretinoin or a salt thereof. Isotretinoin analogs/derivatives have been described in the art and include, e.g., isotretinoin amido alkyl benzoate derivatives (see, e.g., CN103319365) and coenzyme A/N-hydroxysuccinimidyl esters of isotretinoin (see, e.g., EP0271552). Isotretinoin salts include, e.g., zinc salt forms of isotretinoin. Typically, a derivative of isotretinoin does not include or does not include a significant amount of (e.g., more than 5%, more than 2.5%, more than 1.5%, more than 1%, more than 0.5%, more than 0.25%, or more than 0.1% of) either vitamin A or any derivative(s) thereof that is/are substantially less effective than isotretinoin in treating the target disease(s) or condition(s) that the formulation is intended to treat. Any analog/derivative of isotretinoin included in a composition of the invention, or salt of 13-CRA or a 13-CRA analog, should be suitable and effective. In aspects, the isotretinoin compound mostly, generally, substantially, essentially, or entirely consists of isotretinoin base. In aspects, the isotretinoin compound comprises isotretinoin, one or more pharmaceutically acceptable salts thereof, or a combination thereof.

Isotretinoin or other isotretinoin compound(s) of a composition can be present in any suitable amount. In aspects, isotretinoin or one or more salts thereof make up about 1% to about 7% w/w of the total composition. In aspects, isotretinoin or one or more salts thereof make up 3.1% to 6.7% of the composition.

In aspect, the solubilizing agents (i.e., the solubilizing component) make up 50% to 98.5% w/w of the total composition, such as ˜55-99%, 55-95%, 55-97.5%, 60-99%, 60-65%, 60-97.5%, or 65-97.5%, such as ˜62.5-95% or 67.5-97.5% of the composition. In aspects, the solubilizing component makes up at least about 70%, at least about 75%, at least about 80%, at least about 82.5%, at least about 85%, or at least about 90% of the composition.

In aspects, the solubilizing agent is at least mostly composed of, generally consists of, substantially consists of, essentially consists of, or consists of one or more phosphatidylcholines, phosphatidylethanolamines, phosphatidylserines, or phosphatidylinositols, or mixtures thereof. In aspects, a lipid carrier/component of a composition comprises no more than 10%, no more than 7.5%, or no more than 5% of one or each or both of phosphatidylethanolamine (PE) and phosphatidylinositol (PI). In aspects, the main/primary type/class of lipid species/compounds in the composition is/are PC(s). In aspects, PC(s) are among the top 2 or 3 species/classes of lipid compound(s) in a composition.

In aspects, at least about 20%, at least about 25%, or at least about 30%, such about 30% to about 50% or 25-65%, 30-50%, 30-55%, 30-60%, 27.5-55%, 35-55%, 35-60%, 25-55%, or 27.5%-50% of the total composition is composed of one or more phosphatidylcholines (PCs). As is known, phosphatidylcholines (PC) are a class of phospholipids that incorporate choline as a headgroup. Examples of known PCs include 1-oleoyl-2-palmitoyl-phosphatidylcholine and palmitoyl-oleyl-sn-phosphatidylcholine. In aspects, PCs also can include PC derivatives, such as lysophosphatidylcholine(s). In aspects, PCs are limited to non-derivative compound(s). Examples of PC-related compounds are described in, e.g., EP2978416B1. Reportedly about 20-30 PCs are known (see, e.g., Phosphatidylcholine Summary Report prepared for US FDA, prepared by University of Maryland Center of Excellence in Regulatory Science and Innovation, October 2021). In aspects, any suitable PC(s) can be incorporated/used in formulations of the invention.

In aspects, the second solubilizing agent is at least 75% composed of one or more medium chain triglycerides. In aspects, the second solubilizing agent is at least about 85%, at least about 90%, or at least about 95% composed of MCT(s).

In aspects, the composition further comprises a surfactant component, which is at least mostly made up of one or more surfactant(s), optionally with one or more co-surfactant(s), and which surfactant component, in aspects, makes up about 5%-about 35% of the composition (e.g., about 5-45%, about 5-40%, about 10-40%, about 10-35%, about 12.5-37.5%, about 12.5%-27.5%, about 15-35%, about 15-30%, about 15-40%, about 5-25%, about 5-20%, about 10-25%, about 17.5-35%, about 20%, about 25%, about 20-35%, or about 20-40% of the composition). In aspects, the surfactant component can be composed of any one or more suitable surfactant agents/compounds. In aspects, most, generally all, substantially all, essentially all, or all the surfactant component is composed of non-ionic surfactant(s). In aspects, the surfactant component is at least mostly composed of, generally composed of, substantially consists of, essentially consists of, or consists of one or more polyoxyethylene sorbitan fatty acid esters such as one or more polysorbate compounds (e.g., polysorbate 20, polysorbate 80, or a derivative thereof, or combination thereof), one or more caprylocapryol macrogol glycerides, one or more other sorbitan esters (e.g., one or more sorbitan monolaurate(s), such as Span-80 or Span-20), one or more polyoxyl castor oils (e.g., one or more polyoxyl 35 castor oil, polyoxyl 40 castor oil, etc.), or a combination thereof.

In aspects, compositions can also further comprise a suitable number/amount of antioxidant(s). In aspects, antioxidant(s) included in the composition can comprise effective amount(s) of butylated hydroxytoluene, butylated hydroxyanisole, ascorbyl palmitate, propyl gallate, tocopherol compound(s), and combinations thereof. The antioxidants are typically readily lipid/fat soluble. In aspects, antioxidants comprise between 0.01% to 0.03% w/w of the total composition, e.g., about 0.02-0.03%, about 0.015-0.025%, about 0.01-0.02%, or about 0.015%-0.03% of the composition.

The various elements of this disclosure can be combined in any suitable manner. An exemplary composition based upon the various disclosures made herein is a stable, liquid pharmaceutical composition comprising a) 0.1 to 7.0% w/w of isotretinoin (e.g., 0.5-7%, 1-7%, 1.5-7%, 1.5-6.5%, 2-6.5%, 2-6%, 1.75-6%, 1.75-5.75%, 2.5-6%, 2.5-5.75%, 3.1-6.6%, 4.1-6.6%, 3.5-7%, or 4.1-6.2% isotretinoin), b) 0.01 to 0.03% w/w of a pharmaceutically acceptable antioxidant, such as butylated hydroxyl toluene (BHT), c) 5 to 15% w/w of polysorbate(s), such as polysorbate 80, d) 50 to 98.5% w/w of pre-blended phosphatidylcholine with medium chain triglycerides (MCTs), and e) optionally further one or more suitable pharmaceutically acceptable excipients and the mixtures thereof.

In aspects, the liquid pharmaceutical compositions described herein are packaged in plastic barrier bottles, e.g., bottles with Ethylene-vinyl alcohol copolymer (EVOH) or amber colored glass bottles, which can serve to DOS protect compositions stored therein from degradation by light or oxygen.

In aspects, compositions of the invention are characterized in comprising one or more pharmaceutically acceptable excipients selected from one or more colorants, flavors,
artificial sweeteners, chelating agents, preservatives, and mixtures thereof.

In one implementation, the disclosed pharmaceutical composition can be administered orally as such or after mixing with water, milk, and/or soft foods such as ice cream, pudding, whipped cream, oatmeal, yogurt etc. prior to administration.

In one implementation, the present inventors now recognize that the disclosed pharmaceutical composition of isotretinoin is suitable for administration via the nasogastric tube in hospitalized patients unable to take the medication orally.

In aspects, the invention provides a method of treating cancer(s) comprising administering an effective amount of a composition according to any of the disclosures of this section or of this entire disclosure to a subject, such as a patient. Uncontradicted, the term “patient” herein implicitly provides support for a subject. A subject is any suitable non-human mammal, such as a companion animal (e.g., a dog, cat), or an animal that may be used in human clinical studies (e.g., a mouse, dog, rat, rabbit, and the like). A patient is a human patient. Uncontradicted a patient to be treated for a condition may be considered a “patient in need thereof” (i.e., in need of treatment for an indicated disease, condition, or disorder), which is a patient diagnosed as having such a condition, e.g., through a trained professional, diagnostic tool, etc. Treating herein, uncontradicted, in aspects, means to cure, to lessen, to ameliorate, to reduce symptoms of, to reduce duration of, to reduce the extent of, to reduce risks associated with, to improve chances of outcome or condition on outcome of, etc., in a patient, or to achieve such effects in at least a significant number of patients (e.g., most patients, generally all patients, etc.) in a population of patients, such as a sufficient population in one or more well controlled and adequate studies (such as clinical studies that are adequate for FDA approval of a drug).

In aspects, the invention provides a method of treating a dermatological condition comprising acne or rosacea comprising administering an effective amount of a composition according to any of the aspects/embodiments in this section or elsewhere in this disclosure.

In one implementation, the invention provides a method of treating one or more of a dermatological condition, such as Severe Acne Vulgaris, Severe Recalcitrant Nodular Acne, Seborrhea, or rosacea, or one or more other diseases such as Alzheimer's Disease, or more or more cancers, such as those described in the Background or Recurrent Glioblastoma Multiforme (GBM), cervical cancers, Advanced Kidney Cancer, Embryonal Tumors of the Central Nervous System, T-Cell Malignancies, Multiple Myeloma, Malignant Glioma, Relapsed or Refractory Solid Tumors, High-Risk Neuroblastoma, Acute Myeloid Leukemia, Myelodysplastic Syndrome, or Acute Lymphocytic Leukemia, Head and Neck Cancer, and other currently known off-label indications of isotretinoin treatment, by, i.a., administering an effective amount of an oral liquid pharmaceutical composition of isotretinoin compound(s), typically comprising an effective amount of such API(s) solubilized in a phospholipid carrier solution and optionally pharmaceutically acceptable excipients.

In aspects, methods of the invention comprise administering effective amount(s)/number(s) of composition(s) of the invention for the treatment of one or more of acute promyelocytic leukemia, neuroblastoma, medulloblastoma, Cutaneous T-Cell Lymphomas, or squamous cell skin cancers. In aspects, methods of the invention comprise administering effective amount(s) of composition(s) of the invention to treat one or more of basal cell carcinomas, squamous cell carcinomas, superficial bladder tumors and second primary tumors in patients with squamous cell carcinoma of the head and neck. In any of the method aspects described herein, the method can further comprise the combined application of composition(s) of the invention in effective amount(s) in combination with one or more other treatments against the relevant condition (e.g., the use of antibiotic treatment, such as minocycline treatment, for the treatment of acne or rosacea, and the administration of anti-cancer antibodies, checkpoint inhibitors, chemotherapeutic agents, radiation therapy, or surgical methods in the treatment of the various cancers described herein).

In aspects, the invention provides an oral liquid, pharmaceutical composition comprising a therapeutically effective amount of isotretinoin or one or more pharmaceutically acceptable salts thereof, an effective amount of a phospholipid carrier, and an effective amount of one or more other pharmaceutically acceptable excipients.

In aspects, the invention provides an oral liquid pharmaceutical composition according to any one or more of the embodiments/aspects described in this section, wherein the phospholipid component/carrier in the composition ranges from 0.5% to about 97.5% by weight of the composition.

In aspects, the phospholipid component (composed of PC(s) and associated carrier(s), if present, such as where a PC containing mixture is provided as a pre-blended/mixed ingredient) makes up about 25-99%, such as about 33-99%, about 40-99%, about 50-99%, or about 55-99% of the composition. In aspects, the phospholipid component makes up about 40-75% of the composition, such as about 45-70%, about 50-75%, about 55-75%, about 60-75%, about 45-65%, about 50-65%, or about 50-60% of the composition.

In other aspects, the phospholipid carrier/component makes up about 65-99% of the composition, such as about 70-99%, ˜75-99%, ˜80-99%, ˜60-95%, ˜60-90%, ˜70-90%, ˜75-95%, ˜80-95%, ˜55-98%, ˜55-95%, or ˜57-97% of the composition.

In aspects, the phospholipid carrier accounts for at least about 25%, at least about 33%, at least about 50%, at least about 65%, at least about 75%, at least about 80%, at least about 85%, or even at least about 90% of the composition (e.g., about 92.5%, about 95%, or ˜97.5% of the composition).

In aspects, a phospholipid component/carrier can comprise other agents than the phospholipid. In aspects, the invention provides an oral liquid pharmaceutical composition according to any one or more of the embodiments/aspects described in this section, wherein phosphatidylcholine(s) is/are dissolved in a suitable vehicle/carrier system. For example, a phospholipid component/carrier can represent an ingredient comprising effective number(s)/amount(s) of phospholipid(s) in combination with other elements, such as medium chain fatty acids or medium chain triglyceride(s) (MCTs) (e.g., caprylic/capric triglyceride(s)), long chain fatty acids or triglyceride(s) (e.g., oleic acid), alcohol(s) (e.g., ethanol), glyceride(s) (e.g., sunflower seed oil glycerides), antioxidant(s)/preservative(s) (e.g., tocopherol compounds, ascorbyl palmitate, or a mixture thereof). In aspects, the invention provides an oral liquid pharmaceutical composition according to any one or more of the embodiments/aspects described in this section, wherein the phospholipid carrier further is comprised of one or more lipophilic solvents, hydrophilic solvents, emulsifying agents, or combinations thereof.

In aspects, the actual phospholipid content of the phospholipid carrier/component (i.e., the amount of the phospholipid carrier that is composed of phospholipid(s)) is at least about 25%, such as ≥˜33%, ≥˜40%, ≥˜50%, ≥˜65%, or ≥˜70%. In aspects, the actual phospholipid content of the phospholipid carrier/component is at least about 25%, such as ≥˜33%, ≥˜40%, ≥˜50%, ≥˜65%, or ≥˜70% composed of phosphatidylcholine (PC). In aspects, the phospholipid component/content of a formulation generally consists of, substantially consists of, consists essentially of, or consists entirely of phosphatidylcholine (PC).

In aspects, at least about 25%, such as ≥˜33%, ≥˜40%, ≥˜50%, ≥˜65%, or ≥˜70% of a phospholipid carrier of compositions is composed of phospholipid(s), such as, e.g., PC(s). In aspects, the invention provides an oral liquid pharmaceutical composition according to any one or more of the embodiments/aspects described in this section, wherein the phospholipid carrier comprises, mostly comprises, generally consists of, substantially consists of, consists essentially of, or consists of phosphatidylcholine obtained from natural sources such as soybean and egg, synthetic phosphatidylcholine, or both.

In aspects, the invention provides an oral liquid pharmaceutical composition according to any one or more of the embodiments/aspects described in this section, wherein the isotretinoin or salts thereof makes up between 0.1% to 7.0% by weight of the composition, such as about 0.5-6.6% (i.e., 0.5-6.6 wt. %), e.g., about 1-6%, such as about 1.5-5.75%, e.g., about ˜1.75-5.75% (i.e., about 1.75 wt. %-about 5.75 wt. %), such as about ˜1.9-5.6%, ˜2-5.75%, ˜2-6%, ˜2-5.5%, 2-6.5%, or 1.8-5.8%. In aspects, the isotretinoin compound (isotretinoin, isotretinoin salt, or analog/derivative of isotretinoin or a salt thereof) is present in a composition in a weight concentration of about 2%, about 3%, about 4%, about 5%, about 5.5%, or about 5.75% (e.g., about 3-6%, about 3-5.5%, about 3-4.5%, about 2-5%, about 2-4.5%, about 2-4%, about 3.5-6%, about 3.5-5.5%, about 3.5-4.5%, or about 4-6%, about 4-5.5%, or about 4%-5%)

In aspects, the invention provides an oral liquid pharmaceutical composition according to any one or more of the embodiments/aspects described in this section, wherein the composition comprises effective amount(s) of one or more pharmaceutically acceptable excipients selected from one or more of surfactants, antioxidants, chelating agents, preservatives, colors, sweeteners or flavors, and mixtures thereof.

Other objects, features, and advantages of the present invention will become apparent upon consideration of the following detailed description.

### DETAILED DESCRIPTION OF THE INVENTION

For convenience, both combinations of elements/steps and individual elements/steps may be described in this section of this disclosure. Despite the inclusion of passages focused on specific elements/steps, any aspect, facet, embodiment, or other description of particular step(s) or element(s) can be applied to any general description of the compositions/methods of the invention, or any other recited element(s)/step(s) thereof, which are provided in any part of this disclosure.

The present invention relates to a stable oral liquid pharmaceutical composition comprising isotretinoin or salts thereof in solution form which provides flexible and accurate dosing. The process of preparation of these compositions is also provided.

In aspects, liquid pharmaceutical compositions according to the invention are a solution, e.g., a clear solution of isotretinoin or suitable salts thereof, solubilized in a phospholipid carrier, and optionally further containing effective amount(s) of suitable pharmaceutically acceptable excipient(s). The present oral composition of isotretinoin in the form of solution form has the advantage over previously marketed oral compositions of isotretinoin known in the state of the art of having greater flexibility, which allows more accurately adjusting the dose of isotretinoin to the needs of the patient.

Uncontradicted, the term “solution” means a liquid, typically a clear liquid, in which a solute is completely dissolved in a solvent to form a molecularly dispersed system. The primary/principal (main) solute of this invention or at least the primary, if not only, active pharmaceutical ingredient (API), is isotretinoin or salts thereof and the solvent is a phospholipid carrier solution more preferably phosphatidylcholine-based carrier system like those PC compositions currently sold under the PHOSAL® trademark (by Lipoid) (e.g., PHOSAL SA, PHOSAL 50 PG, PHOSAL H 50, PHOSAL Curcumin, PHOSAL 75 SA, PHOSAL MCT, and PHOSAL 53 MCT, or similar/equivalent compositions).

The term “stable” used herein refers to an isotretinoin formulation of the invention, such as an isotretinoin in solution form, which is physically stable according to typical FDA standards of stability, and which typically further also does not significantly precipitate or precipitate any more than a liquid formulation contained in currently approved isotretinoin drug products during storage. Compositions of the invention are, in aspects, stable to oxidation and degradation during storage at recommended storage conditions and under normal or accelerated appropriate FDA stability testing conditions, which are known in the art.

The term “phospholipid carrier” (or phospholipid component) refers to phospholipid molecules in a composition as a carrier solution. Such molecules may be identical to, or different from, each other. In other words, a phospholipid component may comprise a single phospholipid or comprise a mixture of two or more different phospholipids.

The inventors have determined/found that liquid compositions comprising isotretinoin dissolved in phospholipid carrier, such as in a phospholipid solution, can exhibit enhanced solubility and stability as compared to previously described or on-market products.

The content of isotretinoin or a physiologically acceptable salt thereof contained in the pharmaceutical composition of the present invention is generally about 0.1-7% by weight, in aspects about 0.25 to 6.5% by weight (as the converted amount to free isotretinoin) relative to the weight of the composition.

Various lecithin or phospholipid solutions in a suitable vehicle comprising of a lipophilic carrier alone or in combination with hydrophilic carriers may be used in the present oral liquid compositions of isotretinoin.

As is known, phospholipids (PLs) are a class of lipids whose molecule has a hydrophilic “head” containing a phosphate group, and two hydrophobic “tails” derived from fatty acids, joined by an alcohol residue. The phosphate group can be modified with simple organic molecules such as choline, ethanolamine, or serine.

Various phospholipids from plant and animal sources such as soybean phosphatidylcholine, egg phosphatidylcholine, or synthetic phosphatidylcholine, as well as hydrogenated phosphatidylcholine, are commonly used in different types of formulations.

Phospholipids, either alone or mixed with various solvents, can be used as the final ingredient of the formulations mentioned above. These lecithin ingredients can include, for example, lecithin products such as those sold currently under the Alcolec brand, produced by the American Lecithin Company (e.g., ALCOLEC F-100, ALCOLEC S, etc.), Phospholipon®90 brand lecithins (e.g., Phospholipon®90 G, Phospholipon®90 H, other LIPOID P 100 containing lecithins, etc., available from Lipoid GmbH (Germany)), and various types of PHOSAL® ingredients, also produced by Lipoid GmbH which can, in aspects, constitute phosphatidylcholine (PC) with medium chained triglycerides (Phosal®25, 50 & 53 MCT), with propylene glycol (Phosal 50 PG), with safflower oil (Phosal® 50SA+), or with sunflower seed oil (PHOSAL/Phosal® 40 IP), and Lipoid®PPL-600 (soybean phospholipid in soybean oil and medium-chain triglycerides).

Similar compositions, such as compositions comprising most, generally all, substantially all, or all of the same ingredients within +/−˜25%, ˜20%, ˜15%, ˜10%, or ˜5% of the weight percent composition of the ingredient(s) described in any of the ingredient(s) or ingredient formulations provided herein (e.g., the above-described PHOSAL ingredient formulations) can be suitably used/incorporated into compositions of the invention, in aspects.

In aspects, compositions of the invention comprise an ingredient that is a mixture of PC(s) in a carrier system, wherein the PC ingredient comprises about 35-75%, about 40-70%, about 40-65%, about 45-60%, about 47.5-55%, or about 45-55% PC content. In aspects, the PC component comprises both PC and lysophosphatidylcholine (e.g., in a ratio of about 10:1, about 9:1, about 8:1, or about 7:1, 6:1, or 5:1, or range defined by any combination thereof). In aspects, the PC component/carrier system comprises about 2-10%, about 2-8%, about 2.5-7.5%, about 3-7%, about 3-7.5%, about 4-7%, about 4-6.5%, about 5-7%, or about 6% lysophosphatidylcholine, and not less than 50%, not less than 52.5%, not less than 53%, not less than 55%, or not less than 60% PC. In aspects, the PC component/ingredient formulation of a composition/used in the production of a composition DOS lacks lysophosphatidylcholine, or comprises less than about 2%, less than about 1.5%, less than about 1%, or less than about 0.5% thereof. In aspects, the PC carrier system/component has a viscosity of less than about 5000 mPAs at 25° C., such as about 100-5000, about 150-5000, about 500-5000, about 1000-5000, about 1500-5000, or about 2000-5000, e.g., about 2500 or 3000-5000 mPAs. In aspects, the PC component/carrier system further comprises about 1-10% of a suitable alcohol or mixture thereof, such as e.g., ethanol, such as about 2-8%, about 2.5-7.5%, or about 3-6%.

In aspects, the PC solubilizing component carrier (i.e., the carrier of the PC ingredient) further comprises about 10-40%, about 15-35%, about 15-30%, about 20-30%, about 25-30%, or less than about 30%, less than about 25%, or less than about 20% MCT(s). In still other aspects, the PC carrier system can further comprise a high oleic acid content oil, typically at a concentration of less than 10%, less than 7.5%, or less than 5%, e.g., less than 3% or less than 4% (e.g., about 0.1-5%, about 0.25-5%, about 0.5-5%, or about 1-5%). In further aspects, the PC carrier component can comprise oleic acid in a concentration of about 0.1-2.5%, such as about 0.25-2.5%, or about 0.5-2.5%, or any other detectable amount up to about 2.5%, 2.25%, 2%, etc. In aspects, the PC carrier component can further comprise an effective number/amount of antioxidant(s), which typically make up less than 1%, e.g., less about 0.7%, less than about 0.6%, less than 0.5%, or less than about 0.4% of the PC carrier. In aspects, the PC carrier makes up about 50-99%, about 55-98%, about 57.5%-97.5%, about 60-99%, or about 60-95% of the total pharmaceutical composition.

In aspects, a phospholipid component can comprise, e.g., at least about 35%, at least about 42.5%, or at least about 50% PC in combination with an oil component, such as a vegetable oil component, such as a high concentration long chain fatty acid oil/vegetable oil, which maybe a high concentration oleic acid content oil (as described elsewhere), which can be a vegetable oil, such as canola oil, sunflower oil, soybean oil, or modified/concentrated oil, such as a high oleic content or medium oleic content oil, e.g., an oil comprising about 2-20, 3-15, or 3-12% palmitic acid, about 0.1-0.5% or 0.1-0.25% palmitoleic acid, about 1-5%, such as about 2-4% stearic acid, about 50-95%, such as about 55-90% or about 60-90% oleic acid, about 0.5-75%, e.g., about 2-70%, about 2.5-65%, e.g., about 3-60%, or about 5-55% of linoleic acid, about 0.1-1% arachidic acid, such as about 0.1-0.8%, e.g., about 0.2-0.8%, about 0.25-0.75%, or about 0.3-0.75% arachidic acid, about 0.2-2% eicosenoic acid, about 0.1-10%, such as about 0.1-8.5%, e.g., about 0.1-8% or about 2-8% linolenic acid, about 0.2-1.5% or 0.2-1.2% or about 0.3-1% behenic acid, about 0.1-0.5% or 0.2-0.4% lignoceric acid, or a combination of 2, 3, 4, or 5, most, generally all, substantially all, or all thereof. In aspects, a composition comprises an effective amount of an oil (or an oil component comprising oil(s)) that is composed of about 5-20% saturated fats, such as about 6.5-18.5% or 6.5-16.5% saturated fats. In aspects the composition comprises an oil component that is composed of about 80-97.5%, e.g., about 82.5-95%, or about 85-92.5% saturated fats (e.g., about 25-90% or 25-85.5% or 60-90% or 65-95% monosaturated fats, about 2.5-65%, such as about 3-63% polysaturated fats, e.g., about 5-65% or about 5-60% polysaturated fats, or about 15-65% polysaturated fats, or a combination thereof).

The concentration of phospholipid compound(s) present in a composition of the invention can range in aspects from about 0.5% to about 80% by weight, such as about 5% to 75% by weight, for example, about 10% to 65% by weight of the composition. In aspects, the phospholipid compounds make up about 20-55%, e.g., about 22.5-52.5%, about 25-50%, about 30-47.5% or about 30-45% of the composition.

A lipophilic carrier of a composition may include effective/suitable amounts/numbers of fatty acid esters, fatty acids and vegetable oils, and mixtures thereof.

As is known in the art, fatty acid esters are a combination of fatty acid with a polyol, where the fatty acids are specifically medium chain fatty acids and the polyols include glycerol, propylene glycol, polyethylene glycol etc. In aspects, the composition comprises a medium chain fatty acid ester component that is mostly, generally, essentially, substantially, or entirely composed of medium chain triglyceride(s), which are known as medium chain (C6 to C12) fatty acid, such as include caproic acid, caprylic acid, capric acid and lauric acid esters with glycerol. Compositions comprising such MCTs are commercially available under the brand names Labrafac™ lipophile WL 1349, Miglyol® 810, 812, 818 and 829, Crodamol™ GTCC, Captex® 350, 355 and 810D, amongst many others. As with other named excipients recited herein, readers will recognize that suitable substitutes comprising most, generally all, substantially all, essentially all, or all of the same components as these ingredient compositions or equivalent thereof, at concentrations of +/−˜25%, 20%, 15%, 10%, or 5% can be, in aspects, used in compositions of the invention. Fatty acids include saturated or unsaturated fatty acids (C6-C20) such as oleic acid, linoleic acid, caprylic acid or caproic acid. Vegetable oils may be selected from wheat germ oil, soybean oil, olive oil, corn oil, palm oil, coconut oil, groundnut oil, sunflower oil, sesame oil, safflower oil, sweet almond oil, and combinations thereof.

The concentration of the lipophilic vehicle present in the composition can range from about 10% to about 90% wt. In an alternate embodiment of the invention, the concentration of lipophilic vehicle in the composition ranges particularly from about 20% to about 85 wt. %.

The liquid pharmaceutical composition according to the invention may further comprise effective number(s)/amount(s) of hydrophilic vehicles, surfactants, emulsifying agents, antioxidants, chelating agents, sweeteners, flavors, preservatives, and combinations thereof.

A hydrophilic solvent used in compositions of the invention, per aspects, may include, but are not or need not be limited to, monohydric alcohols, glycols, polyols (e.g., polysorbate(s)), and combinations thereof. Examples of hydrophilic carriers that can be utilized/incorporated in aspects are ethanol, propylene glycol, polyethylene glycols, sorbitol, and glycerin, amongst other solvent agent(s)/component(s). The concentration of such agents can range from, e.g., about 0.5% to 50% by weight and particularly about 2% to about 40% by weight of the composition (e.g., about 2-40%, about 4-40%, about 3-30%, about 3-35%, about 3-10%, about 4-20%, about 4-30%, about 5-35%, about 5-50%, about 5-45%, etc.). In aspects, a hydrophilic component can comprise, mostly comprise, generally consist of, substantially consist of, or consist of PEG(s), such as what might be considered a relatively low molecular weight polyethylene glycol (PEG) (e.g., a PEG-8/PEG 400 component, or a PEG component comprising PEG(s) that have a molecular weight (MW) of about 200-800 Daltons, about 300-700, about 200-600, about 300-600, about 300-500, or about 400 Daltons (e.g., 380-420 Daltons)), a polysorbate (e.g., polysorbate 80 or polysorbate 20), or a combination thereof.

In aspects, a composition can further comprise a polyoxyl castor oil (e.g., polyoxyl 35 castor oil, polyoxyl 40 castor oil, or other polyoxyl n castor oil (e.g., where “n” is between 30-40)), e.g., in a concentration of about 5-35%, e.g., about 10-30%, about 12.5-30%, about 15-30%, about 10-25%, about 7.5-25%, about 12.5-25%, about 10-22.5%, about 15-22.5%, or about 17.5-22.5%, such as about 20% of the composition.

In aspects, a hydrophilic solvent portion/component of a composition comprises, mostly comprises, generally consists of, substantially consists of, essentially consists of, or consists of a glyceride composition/ingredient, such as a composition comprising, mostly comprising, generally consisting of, substantially consisting of, essentially consisting of, or consisting of PEG-8 (MW ˜400) mono- and di-esters of caprylic and capric acids (e.g., containing about 10-50%, about 15-45%, or about 15-35% or 20-35% of such an ingredient).

The composition (i.e., a composition of the invention) further may contain effective number(s) and amount(s) of emulsifiers and surfactants, selected from, but not limited to the group consisting of fatty acids, mono- and di-glycerides, sorbitan fatty acid esters, polysorbate derivatives, polyoxyethylene sorbitan fatty acid esters, polyethoxylated castor oils, polyoxyethylene glycol glycerides, propylene glycol esters, fatty acid glycerol esters, Vitamin E TPGS, sodium lauryl sulphate, dioctyl sodium sulfosuccinate, poloxamers and mixtures thereof. Examples include but are not limited to oleic acid, glyceryl monostearate, sunflower seed glycerides, sunflower mono-diglycerides, caprylocaproyl macrogolglycerides, oleoyl polyoxyl-6 glycerides, lauroyl polyoxyl-32 glycerides, stearoyl polyoxylglycerides, D-α-tocopheryl polyethylene glycol succinatem, propylene glycol monocaprylate, span 60, polysorbate 80, poloxamer 188, polyoxyl 35 castor oil (e.g., a composition such as Cremophor EL or a substantially similar/equivalent composition), and polyoxyl 40 castor oil (e.g., a composition such as VitiPure HCO 40 or a substantially similar/equivalent composition).

In embodiments of the invention, emulsifiers and surfactants may be included in the compositions from 1.5% to about 60% by weight. In aspects, the concentration of emulsifier and surfactant is between 2.5% to 50% by weight of the composition (e.g., about 3.5%-65%, about 3.5-55%, about 4%-40%, about 4%-30%, about 3-30%, about 3-35%, about 5%-30%, or about 5-40% of the composition).

The pharmaceutical compositions of the present invention may include effective number(s)/amount(s) of antioxidant(s), such as butylated hydroxy toluene (BHT), butylated hydroxy anisole (BHA), ascorbyl palmitate, ascorbic acid, propyl gallate, tocopherol, sodium sulfite, sodium metabisulfite, sodium thiosulfate, etc.

Chelating agents like disodium edetate, calcium disodium edetate, citric acid, tartaric acid may also be incorporated in the disclosed pharmaceutical compositions.

Suitable other preservatives that may be used/included in the compositions of the present invention can include methyl paraben, ethyl paraben, propyl paraben, butyl paraben, sodium benzoate, and combinations thereof.

Examples of sweeteners that can be used in compositions of the invention can include sorbitol, mannitol, xylitol, saccharin, sodium or calcium saccharin, aspartame, acesulfame potassium, sodium cyclamate, sucralose, or a combination thereof. Coloring agents and flavoring agents may be selected from any FDA approved colors and flavors for oral use.

The isotretinoin compositions disclosed in the present invention may further comprise lipophilic or hydrophilic pharmaceutical excipients alone or in appropriate combination which are soluble or miscible with the phospholipid carrier phase and enhance the physical, chemical, or microbiological stability of the composition.

In aspects, any 2, 3, 4, or 5 of the above-described ingredients or classes of ingredients (e.g., the isotretinoin, the lipid solubilizing agent (e.g., a PC solubilizing agent component or PC), a stabilizing component (e.g., a surfactant component)) can constitute most, generally all, substantially all, essentially all, or all of the liquid composition in which the isotretinoin compound(s) are formulated. E.g., such a combination of ingredients or components can make up at least about 65%, 80%, 85%, 92.5%, 97.5%, 99%, or 100% of the liquid composition in which the isotretinoin compound(s) are contained.

According to aspects, any component(s) or compound(s)/agent(s) described herein can be present in composition(s) in therapeutically effective amount(s), compositionally compatible amount(s), or both. In aspects, any single component or compound/agent provided herein can be present in a relationship with, such as, e.g., in a ratio with, any one or more other single component or compound/agent. In aspects, any combination of component(s) or compound(s)/agent(s) provided herein can be present in a ratio with any other combination of component(s) or compound(s)/agent(s). In aspects, ratio(s) between such component(s) or compound(s)/agent(s) or combinations thereof can be established using any provided amounts for each disclosed herein, including, e.g., values within ranges of such amounts disclosed herein.

In an aspect, compositions of the invention can be characterized by comprising an effective amount of isotretinoin, and an effective amount of PC(s), wherein the isotretinoin component is present in a ratio of about 1:3 to 1:50, such as about 1:4 to about 1:40, e.g., about 1:4 to about 1:30 or about 1:3 to about 1:30, e.g., about 1:5 to 1:20 or about 1:5 to 1:15. In aspects, the composition comprises at least about 1%, e.g., at least about 1.5% or at least about 2% isotretinoin or other isotretinoin compound. In aspects, the composition also or alternatively comprises at least about 15%, e.g., at least about 20%, or at least about 25% PC.

In an aspect, compositions according to the invention can be characterized based on comprising an effective amount of isotretinoin/isotretinoin compounds, an effective amount of PC, an effective amount of a polysorbate surfactant, wherein the polysorbate surfactant (e.g., polysorbate 80) is present in a ratio to the amount of PC of about 1:1.5 to about 1:12, e.g., about 1:2 to about 1:10 or about 1:3 to about 1:9. In aspects, compositions comprise a surfactant component comprising relatively low MW PEG(s), e.g., PEG200-PEG800, PEG200-PEG600, or PEG200-PEG400, and a polysorbate, wherein the ratio of isotretinoin to the PEG/polysorbate surfactant component is about 1:4 to about 1:20, such as about 1:3 or 1:4 to about 1:15, such as about 1:1.75 to 1:2.5, such as about 1:2, 1:3, about 1:4, or about 1:5.

In aspects, compositions of the invention comprise a polyoxyl castor oil component, in addition to effective amounts of isotretinoin and PC, wherein the ratio of the isotretinoin to the polyoxyl castor oil (PCO) is about 1:3 to about 1:15, e.g., 1:4 to 1:16 or 1:4 to 1:12 or 1:3 to 1:9, 1:4 to 1:10, or 1:4 to 1:12. In aspects, compositions comprise at least about 10%, at least about 12.5%, at least about 15%, or at least about 20%, such as about 10-25%, 12.5-25%, or 15-25% PCO(s), e.g., about 17.5-30% PCO, or about 17.5-25% PCO. In aspects, the PCO component/ingredient of a composition mostly comprises, generally consists of, substantially consists of, consists essentially of, or consists entirely of polyoxyl 35 castor oil.

In aspects, compositions of the invention includes both an effective amount of a PCO and an effective mount of a polysorbate, in addition to the isotretinoin and phospholipid (e.g., PC) components. In aspects, the combination of PCO/polysorbate to PC is present in a ratio of about 1:1.5 to about 1:4, 1:3.5, or 1:3. In aspects the combination of PCO/polysorbate to the PC is present in a ratio of about 1:1.75 to 1:2.5, e.g., about 1:2.

In aspects, compositions of the invention include PEGs, e.g., relatively low MW PEGs, e.g., PEGs of from PEG-200 to PEG-600, such as about PEG-400 in size, wherein the ratio of isotretinoin to the PEG component is about 1:3 to about 1:20, such as about 1:4 to about 1:16 or 1:4 to about 1:12, such as about 1:2 and about 1:2.5.

In aspects, compositions of the invention comprise a high oleic acid content oil (HOACO). In aspects the HOACO is a vegetable oil, such as a sunflower oil, safflower oil, or mixture thereof. In aspects, the HOACO is present in an amount such that the ratio of isotretinoin to the HOACO is about 1:3 to about 1:15, such as 1:4 to 1:16 or 1:4 to 1:12, e.g., about 1:5 to 1:12 or 1:5 to about 1:10. In aspects, the ratio of the HOACO to PC in the composition is about 1:1.5 to 1:3.5, e.g., about 1:2 to about 1:3 or about 1:2 to about 1:2.5. The characteristics of exemplary oils that constitute HOACOs and can be incorporated in compositions are described elsewhere herein. In aspects, the HOACO comprises about 55-90% oleic acid, e.g., about 60-85% oleic acid. In aspects, the HOACO is further characterized in comprising about 0.5-60% linoleic acid; about 1-5% such as about 2-4% stearic acid; about 3-12% such as about 4-11% palmitic acid; or a combination of any or all thereof.

In aspects, compositions of the invention can be characterized by lacking one or more characteristics of the compositions described in the art incorporated herein by reference.

In aspects, compositions of the invention comprise no more than 20% macrogol glycerides, e.g., no more than 18% or no more than 15% macrogol glycerides.

In aspects, compositions of the invention comprise less than 70%, less than 60%, less than 55%, or less than 50% MCT content (e.g., only comprising 20-45%, 25-45%, 27.5-47.5%, 25-35%, 27.5-37.5%, 22.5%-37.5%, or about 30% MCT content).

In aspects, about 15%, about 20%, about 25%, or about 30% of the composition is made up of lipids having an HLB value of less than about 4, such as less than about 3, or less than about 2 (e.g., about 1-4, 1-3, 1-2, or about 1).

In aspects, a composition of the invention comprises a surfactant component mostly, generally, substantially, or entirely made up of compounds that have an HLB of at least 11, such as 11-16, e.g., about 12-15.

In aspects, the compositions of the invention can be characterized as comprising a solubilizer component. As described elsewhere, the solubilizer component typically comprises an effective amount of lipid(s), in aspects comprising an effective amount/number of phospholipids, and comprising, according to more particular aspects, an effective amount/number of PC(s). The compositions of the invention also typically can be characterized as comprising a stabilizer component, comprising a high oleic acid content oil, PEGs, PEG/MCT mixtures, polysorbates, POCs, or a combination thereof.

According to aspects, compositions can comprise at least 3% isotretinoin, e.g., at least about 3.3%, 3.5%, or 3.75% isotretinoin, such as 3.1-7.5%, 3.3-7.3%, 3.5-7%, 3.1-6.5%, 3.3-6.6%, 3.2-5.7%, 3.3-5.8%, 3.5-5.5%, or 3.5-6% isotretinoin.

According to aspects, compositions can be characterized as having an MCT content of less than 65%, less than 50%, or even less than 40%. In aspects, the total amount of a composition also or alternatively is at least 10%, at least 15%, at least 20%, or at least 25% composed of MCT(s). In aspects, compositions are characterized in having an ISO to MCT ratio of less than about 1:150, such as less than 1:100, less than 1:75, or less than about 1:50 (e.g., less than 1:30 or less than ˜1:25).

In embodiments, compositions can be characterized in having a propylene glycol (PG) compound content that is less than the MCT content of the composition. In aspects, the propylene glycol compound content of compositions is less than about 65%, less than 50%, or less than about 40% of the total composition (in this and similar respects, uncontradicted, the total composition referred to can mean the total liquid composition containing the isotretinoin compound(s) and is not intended to, e.g., encompass a capsule comprising such a composition, other agents that may be combined with the composition, etc.). In aspects, the ratio of ISO (isotretinoin compound/isotretinoin) to PG compound(s) (e.g., propylene glycol monolaurate) in a composition is less than about 1:150, such as less than 1.100, less than 1:75, or less than 1:50.

As noted, the inventors have determined through observation or inventive insight that the inclusion of effective amounts of lipids, such as phospholipids, in formulations of the invention can lead to unexpected improvements in terms of solubility and stabilization of compositions as compared to other compositions, including compositions such as currently marketed ISO formulations and those described in the prior art references incorporated herein.

Compositions of the present inventions may be prepared using any suitable number of processes known to persons ordinarily skilled in the field of pharmaceutical manufacture. According to one of the embodiments, a composition of the present invention is prepared by dissolving an effective and suitable number/amount of isotretinoin compound(s), such as isotretinoin, in a lipophilic phospholipid carrier by continuous stirring at room temperature or higher temperature of about 45-50° C. to detectably minimize temperature associated degradation, although in aspects even higher temperatures can also be used in such a production method. Additionally, in aspects a composition is processed in a room/area or container where sodium vapor lamp is the source of light, and an inert gas (such as nitrogen) is purged through the solution during manufacturing to protect the isotretinoin from oxidation due to atmospheric air or air entrapped in the carrier system. Such methods can be combined with any of the details/steps in the exemplified production method included in the Examples. Compositions produced by such methods are a further aspect of this invention (e.g., the method provides products produced by such methods).

According to another embodiment, a composition of an isotretinoin compound composition/solution may be packed in suitable primary packaging made of glass or suitable plastics, which provides an oxygen and light barrier, and can store isotretinoin solution throughout the intended shelf life of the product. The packaging may be closed, e.g., with a suitable child resistant closure, and the headspace in the bottle may be filled with an inert gas, such as nitrogen. The liner material of closure that contacts the isotretinoin compound solution may be made of compatible materials such as expanded polyethylene, thin aluminum strip or any other non-reactive and non-shedding materials for maintaining stability of the composition.

According to another embodiment, the package may be supplied with a calibrated dosing device such as droppers, medication cup, dosing spoon, oral dosing syringe along with a neck adaptor for accurate measurement of the doses. The package also can provide for flexible dosing of the pharmaceutical solution in addition to protecting the caregivers from exposure to the medication.

The composition of the present invention may be administered as such or after mixing with liquids such as water, milk, and/or soft foods such as ice cream, pudding, oatmeal, butter, yogurt etc. prior to administration. Further, the composition is suitable for administration via the nasogastric tube in hospitalized patients unable to take the medication orally.

The present invention also relates to methods of treatment of high-risk neuroblastoma and severe recalcitrant nodular acne by administering a therapeutically effective dose of the composition of the present invention to a patient suffering from the applicable disorder. Other diseases which include disorders/conditions that are treatable through isotretinoin treatment, including but not limited to diseases such as seborrhea, recurrent glioblastoma multiforme (GBM), cervical cancers, advanced Kidney cancer, embryonal tumors of the central nervous system, T-cell malignancies, multiple myeloma, malignant glioma, relapsed or refractory solid tumors, acute myeloid leukemia, myelodysplastic syndrome, acute lymphocytic leukemia, head and neck cancer, acne vulgaris, pyoderma faciale, psoriasis, cutaneous lupus erythematosus, acne fulminans, and generalized lichen planus, may also be treated by administering effective amount(s) of a pharmaceutical solution of the invention comprising isotretinoin or pharmaceutically acceptable salts thereof, an effective lipid/phospholipid carrier, and optionally pharmaceutically acceptable excipients.

Any of the elements described herein can be characterized as “means for” performing an indicated function (for compositions) or “steps for” carrying out a method. For example, a composition of the invention can be characterized as comprising a surfactant means, an antioxidant means, a solubilizing means comprising (e.g., mostly comprising PC), and the like. A method can comprise means for storing compositions, steps for administering compositions, and the like.

### Representative Exemplary Embodiments (“Examples”)

The following detailed exemplary embodiments, of or otherwise related to the invention (“Examples”) are provided to assist readers in further understanding aspects of the invention or principles related to the invention or practice of aspects of the invention.

Any particular materials, methods, steps, ingredients, amounts, and conditions employed/described in the following Examples, and any results thereof, are merely intended to further illustrate aspects of the invention. These Examples reflect exemplary embodiments of the invention, and the specific methods, findings, principles of such Examples, and the general implications thereof, can be combined with any other part of this disclosure. Readers should understand that the invention is not limited by these Examples in totality or any part thereof.

### Example 1

A first exemplary composition of the invention is prepared having the following composition:

| S. No | Composition | % |
|---|---|---|
| 1 | Isotretinoin | 5.7 |
| 2 | Butylated hydroxyl Toluene | 0.02 |
| 3 | PHOSAL 53 MCT | 94.28 |

The composition is expected to be useful as an alternative formulation for the effective delivery of isotretinoin compounds/isotretinoin. This Example exemplifies that liquid pharmaceutical compositions of the invention can be characterized as comprising, substantially consisting of, or consisting essentially of an isotretinoin compound, a phospholipid component that is at least mostly composed of phosphatidylcholine, and a suitable antioxidant or preservative, without the inclusion of other elements/ingredients. This Example also demonstrates that the invention provides compositions wherein about 50% of the composition can be composed of PC(s). Moreover, this exemplary formulation exemplifies that compositions of the invention can be characterized on, i.a., the lack of various components, such as glycerides, PEGs, and the like or by MCTs making up less than about 70%, less than about 60%, or less than about 50% of the composition. These points serve to set such and similar compositions of the invention apart from the previous attempts to generate effective oral liquid formulations of isotretinoin, such as those attempts described in the references cited in the Background of this disclosure and incorporated herein by reference.

### Example 2

Another exemplary formulation of the invention has the following composition:

| S. No | Composition | % |
|---|---|---|
| 1 | Isotretinoin | 2.0 |
| 2 | Butylated hydroxyl Toluene | 0.02 |
| 3 | PHOSAL50 PG | 97.98 |

This composition has many/most of the features/characteristics described above in connection with the exemplary formulation of Example 1, but at an even higher concentration of PC(s).

### Example 3

A further exemplary composition of the invention has the following composition:

| S. No | Composition | % |
|---|---|---|
| 1 | Isotretinoin | 2.0 |
| 2 | Butylated hydroxyl Toluene | 0.02 |
| 3. | Polysorbate 80 | 10.0 |
| 3 | PHOSAL 50SA+ | 87.98 |

This Example exemplifies a composition in which the applicable formulation comprises a surfactant (e.g., a mostly, generally, or entirely non-ionic surfactant) component, here being a polysorbate component (specifically, polysorbate 80). The Example exemplifies compositions consisting or consisting essentially of a PC component, an antioxidant/preservative, isotretinoin, and a surfactant component. Such compositions are expected to provide useful alternatives to known or previously proposed formulations of isotretinoin.

### Example 4

This Example describes still another exemplary composition of the invention.

| S. No | Composition | % |
|---|---|---|
| 1 | Isotretinoin | 5.3 |
| 2 | Butylated hydroxyl Toluene* | 0.02 |
| 3 | Tween 80 | 6.6 |
| 4 | PHOSAL 53 MCT | 88.08 |

This exemplary composition exemplifies that the amount of non-ionic surfactant, tretinoin, and PC component of inventive formulations can be varied and still be expected to result in formulations with novel and inventive properties with respect to previously described or marketed isotretinoin products. Accordingly, consistent with the disclosure provided above, the invention provides a class of formulations that are characterized by various ingredients/components or classes of ingredients/components, in effective amounts.

### Example 5

This Example sets forth still a further exemplary pharmaceutical formulation according to the invention:

| S. No | Composition | % |
|---|---|---|
| 1 | Isotretinoin | 4.0 |
| 2 | Butylated hydroxyl Toluene* | 0.03 |
| 3 | Caprylocaproyl macrogol glycerides | 30.0 |
| 4 | PHOSAL 53 MCT | 65.97 |

This Example illustrates formulations comprising a separate PEG/MCT surfactant component (caprylocaproyl macrogol glycerides, a type of caprylocaproyl polyoxyglyceride composition). Here the exemplified non-ionic surfactant component makes up 30% of the total composition. Such compositions are expected to exhibit useful properties with respect to their isotretinoin active pharmaceutical ingredient content and applications.

### Example 6

This Example illustrates still another exemplary composition of the invention. In this case, as exemplified, the composition comprises a surfactant component composed of both a polysorbate agent (here, polysorbate 80) and a second PEG/MCT surfactant component, together making up 30% of the total composition, and being present in an approximately 1:5 ratio, the PEG/MCT component further being present in a ratio with the isotretinoin of the composition of 1:6.25.

| S. No | Composition | % |
|---|---|---|
| 1 | Isotretinoin | 4.0 |
| 2 | Butylated hydroxyl Toluene* | 0.03 |
| 3 | Polysorbate 80 | 5.0 |
| 4 | Caprylic and capric acid triglycerides | 25.0 |
| 5 | PHOSAL 53 MCT | 65.98 |

Such compositions are expected to exhibit useful properties/characteristics in the practice of various methods of the invention.

### Example 7

This Example provides yet another exemplary composition according to the invention as specified:

| S. No | Composition | % |
|---|---|---|
| 1 | Isotretinoin | 3.0 |
| 2 | Butylated hydroxyl Toluene | 0.03 |
| 3 | Polyoxyl 35 castor oil | 20.0 |
| 4 | PEG 400 | 20.0 |
| 5 | PHOSAL 53 MCT | 56.97 |

This composition includes a polyoxyl castor oil surfactant component in addition to a PEG 400 component. The two components make up a surfactant component that represents 40% of the total composition. Thus, this composition also illustrates embodiments in which the surfactant component (40%) is at a greater concentration than the PC ingredient(s) of the composition (here, about 30% PC to about 40% surfactant, representing a 3:4 ratio). Such compositions are expected to exhibit useful properties for the medical delivery of isotretinoin.

### Example 8

This Example describes a further exemplary composition of the invention—

| S. No | Composition | % |
|---|---|---|
| 1 | Isotretinoin | 2.0 |
| 2 | Butylated hydroxyl Toluene | 0.03 |
| 3. | Polyethylene glycol 400 | 20.0 |
| 3. | Polysorbate 80 | 10.0 |
| 3 | PHOSAL 53 MCT | 67.97 |

The exemplified composition includes both a PEG 400 and polysorbate 80 surfactant component, here making up (collectively) 30% of the formulation. Thus, this composition reflects embodiments in which the PC content of the formulation is in excess of the surfactant content. Compositions having these and similar features are expected to be useful in the delivery of tretinoin and in the practice of methods described herein.

### Example 9

This Example sets forth a final exemplary composition of the invention as depicted in the following table—

| S. No | Composition | % |
|---|---|---|
| 1 | Isotretinoin | 4.0 |
| 2 | Butylated hydroxyl Toluene* | 0.03 |
| 3 | Polysorbate 80 | 5.0 |
| 4 | Soyabean oil | 25.0 |
| 5 | PHOSAL 53 MCT | 65.98 |

This Example demonstrates formulations that include a high oleic acid content oil as a major component of the formulation (here, soyabean oil). The exemplified composition further comprises a polysorbate surfactant (polysorbate 80 at 5%). Such alternative formulations are expected to exhibit useful properties in the delivery of isotretinoin and for use in methods of the invention described herein.

### Example 10

This Example describes one exemplary general procedure for the manufacture of formulations according to the invention.

A procedure for the mixing of the ingredients of a formulation of the invention is carried out under sodium vapor lamp and continuous nitrogen purging.

After dissolving the antioxidant in a PHOSAL PC carrier, isotretinoin is dissolved in the formulation/system under continuous stirring. After the API is completely dissolved and a clear solution is formed, the remainder of the ingredients can be added.

The resulting composition(s) can be filled into capsules as such or after adsorbing it on excipients such as silicon dioxide, lactose, microcrystalline cellulose, magnesium aluminosilicate, etc.

This Example demonstrates how known methods can be combined in the generation of compositions of the invention to result in compositions that are expected to be pharmaceutically useful/suitable.

What is claimed is:

1. A stable, liquid pharmaceutical composition comprising (1) a therapeutically effective amount of an isotretinoin compound; (2) a solubilizing component that is at least 50% composed of an effective amount of a mixture of (a) a first solubilizing agent composed of phosphatidylcholine and (b) a second solubilizing agent that is at least mostly composed of (I) a pharmaceutically acceptable medium chain triglyceride or mixture of two or more pharmaceutically acceptable medium chain triglycerides, (II) a high oleic acid content oil comprising about 25-90% oleic acid, (III) a propylene glycol, or (IV) a combination thereof, wherein (3) the (a) amount of phosphatidylcholine in the solubilizing component is greater than the amount of the second solubilizing agent, (b) the phosphatidylcholine makes up at least 25% w/w of the total composition, or (c) the amount of phosphatidylcholine in the solubilizing component is greater than the amount of the second solubilizing agent and the phosphatidylcholine makes up at least 25% w/w of the total composition; and (4) a stabilizer component, comprising an effective amount of one or more stabilizing agents, wherein the one or more stabilizing agents comprise one or more antioxidants, surfactants, medium chain triglycerides, or mixtures thereof, wherein the composition is a liquid at room temperature under ordinary storage conditions.

2. The liquid pharmaceutical composition as claimed in claim 1, wherein the isotretinoin compound comprises isotretinoin, one or more pharmaceutically acceptable salts thereof, or a combination thereof.

3. The liquid pharmaceutical composition as claimed in claim 2, wherein the isotretinoin or one or more salts thereof makes up about 1% to about 7% w/w of the total composition.

4. The liquid pharmaceutical composition as claimed in claim 3, wherein the isotretinoin or one or more salts thereof makes up 3.1% to 6.7% w/w of the composition.

5. The liquid pharmaceutical composition as claimed in claim 1, wherein the solubilizing component makes up 50% to 98.5% w/w of the total composition.

6. The liquid pharmaceutical composition as claimed in claim 3, wherein the solubilizing component makes up 50% to 98.5% w/w of the total composition.

7. The liquid pharmaceutical composition as claimed in claim 4, wherein the solubilizing component makes up 50% to 98.5% w/w of the total composition.

8. The liquid pharmaceutical composition as claimed in claim 5, wherein about 30% to about 50% w/w of the total composition is composed of one or more phosphatidylcholines.

9. The liquid pharmaceutical composition as claimed in claim 6, wherein about 30% to about 50% w/w of the total composition is composed of one or more phosphatidylcholines.

10. The liquid pharmaceutical composition as claimed in claim 7, wherein about 30% to about 50% w/w of the total composition is composed of one or more phosphatidylcholines.

11. The liquid pharmaceutical composition as claimed in claim 8, wherein the second solubilizing agent is at least 75% composed of one or more medium chain triglycerides.

12. The liquid pharmaceutical composition as claimed in claim 9, wherein the second solubilizing agent is at least 75% composed of one or more medium chain triglycerides.

13. The liquid pharmaceutical composition as claimed in claim 10, wherein the second solubilizing agent is at least 75% composed of one or more medium chain triglycerides.

14. The liquid pharmaceutical composition as claimed in claim 11, wherein the composition comprises a surfactant component that makes up about 5%-about 35% w/w of the composition.

15. The liquid pharmaceutical composition as claimed in claim 12, wherein the composition comprises a surfactant component that makes up about 5%-about 35% w/w of the composition.

16. The liquid pharmaceutical composition as claimed in claim 13, wherein the composition comprises a surfactant component that makes up about 5%-about 35% w/w of the composition.

17. The liquid pharmaceutical composition as claimed in claim 14, wherein the surfactant component is at least 50% composed of one or more polyoxyethylene sorbitan fatty acid esters, one or more caprylocapryol macrogol glycerides, one or more sorbitan monolaurates, one or more polyoxyl castor oils, or a combination thereof.

18. The liquid pharmaceutical composition as claimed in claim 15, wherein the surfactant component is at least 50% composed of one or more polyoxyethylene sorbitan fatty acid esters, one or more caprylocapryol macrogol glycerides, one or more sorbitan monolaurates, one or more polyoxyl castor oils, or a combination thereof.

19. The liquid pharmaceutical composition as claimed in claim 16, wherein the surfactant component is at least 50% composed of one or more polyoxyethylene sorbitan fatty acid esters, one or more caprylocapryol macrogol glycerides, one or more sorbitan monolaurates, one or more polyoxyl castor oils, or a combination thereof.

20. A method of treating cancer comprising administering an effective amount of a liquid pharmaceutical composition according to claim 11 to a cancer patient.

21. A method of treating a dermatological condition comprising acne or rosacea comprising administering an effective amount of a liquid pharmaceutical composition according to claim 11 to an acne or rosacea patient.

22. A method of treating cancer comprising administering an effective amount of a liquid pharmaceutical composition according to claim 12 to a cancer patient.

23. A method of treating a dermatological condition comprising acne or rosacea comprising administering an effective amount of a liquid pharmaceutical composition according to claim 12 to an acne or rosacea patient.

24. A method of treating cancer comprising administering an effective amount of a liquid pharmaceutical composition according to claim 13 to a cancer patient.

25. A method of treating a dermatological condition comprising acne or rosacea comprising administering an effective amount of a liquid pharmaceutical composition according to claim 13 to an acne or rosacea patient.

26. A method of treating cancer comprising administering an effective amount of a liquid pharmaceutical composition according to claim 17 to a cancer patient.

27. A method of treating a dermatological condition comprising acne or rosacea comprising administering an effective amount of a liquid pharmaceutical composition according to claim 17 to an acne or rosacea patient.

28. A method of treating cancer comprising administering an effective amount of a liquid pharmaceutical composition according to claim 18 to a cancer patient.

29. A method of treating a dermatological condition comprising acne or rosacea comprising administering an effective amount of a liquid pharmaceutical composition according to claim 18 to an acne or rosacea patient.

30. A method of treating cancer comprising administering an effective amount of a liquid pharmaceutical composition according to claim 19 to a cancer patient.

31. A method of treating a dermatological condition comprising acne or rosacea comprising administering an effective amount of a liquid pharmaceutical composition according to claim 19 to an acne or rosacea patient.
